(19) United States
(12) Patent Application Publication
(10) Pub. No.: US 2025/0332117 A1
(43) Pub. Date: Oct. 30, 2025

(54) Thin Films with Peroxides and Uses Thereof

(71) Applicant: Silverpeutics, Inc., Livermore, CA (US)

(72) Inventors: Kevin Krogman, Brookdale, CA (US); Victoria Piunova, Brookdale, CA (US)

(73) Assignee: Silverpeutics, Inc., Livermore, CA (US)

(21) Appl. No.: 19/184,429

(22) Filed: Apr. 21, 2025

Related U.S. Application Data
(60) Provisional application No. 63/638,083, filed on Apr. 24, 2024.

(51) Int. Cl. A61K 9/70; A61K 33/40; A61K 47/18; A61K 47/32; B29C 41/00; B29C 41/26; B29C 41/44; B29K 29/00; B29K 105/00; B29L 31/00

(52) U.S. Cl. CPC A61K 9/7007; A61K 33/40; A61K 47/18; A61K 47/32; B29C 41/003; B29C 41/26; B29C 41/44; B29K 2029/04; B29K 2105/0011; B29K 2995/0092; B29L 2031/753

(57) ABSTRACT

The present disclosure provides a thin film made from a hydrophilic polymer, where the thin film includes a peroxide and/or an additive, methods of for making these thin films, and methods of using these thin films.

### BACKGROUND

Appropriate and optimal wound care is crucial, especially in the presence of pathology. Increased prevalence of obesity and diabetes further emphasize the benefit and need for improved wound care. A recent study revealed that wound healing is facilitated by a moist dressing. Moist wound healing is a therapeutic method with exudate as the humectant to protect and provide a moist environment for the wound. Cellulose materials such as a bandage or gauze is used in clinical wound care.

Skin wound healing involves multiple processes: (1) hemostasis, (2) inflammation, (3) proliferation, and (4) remodeling. Inflammation is a tissue defense mechanism, and provides resistance to microbial contaminations. Inflammation occurs almost simultaneously with hemostasis, and starts from within a few minutes to 24 h from injury and lasts for about 3 days. Proliferation starts at approximately day 3, in which keratinocytes and fibroblasts start to proliferate and migrate toward the wound. Failed regulation of any particular process results in pathologically compromised wound healing, such as chronic wounds, which are characterized by a prolonged or excessive inflammatory phase, persistent infections, and delayed wound contraction.

During the inflammatory phase, vascular contraction increases vascular permeability, allowing neutrophils, macrophages, and lymphocytes to invade. Cell proliferation is the next stage, and it is generally acknowledged that fibroblasts are necessary for both cell proliferation and the development of new blood vessels since they release collagen. During the remodeling phase, collagen near the injury site is reorganized, and angiogenesis activity is stopped. Additionally, collagenase then mediates the recycling of collagen, with too much collagen at the injury site eventually leading to the creation of hard scars.

### SUMMARY

The disclosure here relates to thin films made from hydrophilic polymers such as polydiallyldimethyl ammonium chloride, polyacrylic acid, sulfonated polystyrene, chitosan, chitin, carboxymethylcellulose, hyaluronic acid, polyvinylpyrrolidone, polyvinyl alcohol, polyallylamine, polythiophenes, polyethyleneimines, polyacrylamides or copolymers or combinations thereof. In an aspect, the hydrophilic polymer is GRAS (generally regarded as safe) including, for example, polyvinyl alcohol (PVA), polysaccharides, chitosan, starch, alginate, dextran, dextrin, chitin, guar gum, gum karaya, agar, Fenugreek seed mucilage, Soy polysaccharide, Gellan gum, Mango peel pectin, Lepidium sativum mucilage, Plantago ovata seed mucilage, Aegle marmelos gum, Locust bean gum, Lepidium sativum, Mangifera indica gum, Hibiscus rosa-sinensis mucilage, carrageenan, hyaluronic acid, carboxymethylcellulose, carnauba wax, carob bean gum, carotene, cellulose, gelatin, gum Arabic, gum Ghatti, gum gualac, gum tragacanth, hydroxypropylmethyl cellulose, methylcellulose, polyethylene glycol (PEG), propylene glycol.

The thin films can contain agents that kill microorganisms or inhibit the growth of microorganisms. In an aspect, the thin film has a peroxide and/or an additive included in the film. The peroxide can be any peroxide including, for example, hydrogen peroxide, calcium peroxide, sodium peroxide, lithium peroxide, barium peroxide, magnesium peroxide, zinc peroxide, carbamide peroxide, benzoyl peroxide (e.g., for acne treatment), and hydrogen peroxide-urea. The additive can be any beneficial agent including, for example, salicylic acid, hydroquinone, retinoids, hyaluronic acid, and/or vitamin C.

Thin films of the disclosure can be made by using the wet coating techniques to make a layer or layers of polymer(s), after which the wet film can be dried. Applicable wet coating techniques include, for example, reverse-roll coating, knife-over-roll coating, Meyer rod coating, gravure, slot-die, dip coating, spin coating, and spray coating. Other methods for making the thin films herein include, for example, immersion, inkjet, flexographic, metering rod, blade, air knife, curtain, melt extrusion, solvent casting and any combinations of the methods described above.

In an aspect, thin films described herein can be used as a vehicle delivering desired levels of peroxide to aid or assist in wound healing. The thin films described herein can be incorporated into existing wound care therapies to add complimentary benefits. In an aspect, the thin film may be made of PVA (polyvinyl alcohol). Such PVA thin films can dissolve and bio-resorb while delivering a payload included in the thin film. Exemplary payloads include, for example, peroxides such as urea percarbonate or sodium percarbonate. Other additives can also be included in the thin film for delivery at the site of application.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

As used in this specification and the appended claims, the singular forms “a”, “an” and “the” include plural referents unless the context clearly indicates otherwise. Thus, for example, reference to “a polypeptide” includes more than one polypeptide.

The section headings used herein are for organizational purposes only and not to be construed as limiting the subject matter described.

### Definitions

As used herein and unless otherwise specified, the terms “coating” and “film” are used interchangeably.

As used herein, the terms “hard” “durable” and “durability” refer to the ability of a coating material to resist a stress or force, possess increased toughness, viscosity, modulus, or other material properties known in the art, or to resist deterioration, damage or degradation during a predetermined period of time, e.g., the lifetime of the material. The durability of a coating material may be characterized by its ability to maintain one or more properties of the material, such as but not limited to, appearance, strength, or an optical property (e.g., reflectance or haze). Appearance may be assessed by the observation of defects such as cracks, wrinkles and fogging. Strength may be assessed by any convenient standard test, e.g., the pencil test for film hardness (ISO 15184). In a durable coating such as those of the invention, such properties may be maintained over an extended period of time, such as, 1 day or more, 1 week or more, 1 month or more, 2 months or more, 3 months or more, 4 months or more, 5 months or more, 6 months or more, 12 months or more, 18 months or more, or even 24 months or more.

As used herein unless indicated otherwise, the term “ionic moieties” is meant to include moieties that are electrostatically charged at any pH (e.g., hard quaternary ammonium moieties), moieties that are electrostatically charged only at certain pH (e.g., primary, secondary, and tertiary amine moieties, carboxylic acids, etc.), and “ionizable moieties” (i.e., moieties that can be converted to an ionic moiety via a hydrolysis or substitution reaction). Examples of ionic moieties are amines (i.e. primary, secondary, tertiary, and quaternary amines), hydroxyl (including protected hydroxyl such as alkoxy and aryloxy), amides, thiol, acids (e.g., sulfinic acid), sulfinates, silanols, and carboxylic acid (including protected carboxylic acids such as carboxylates) and the like.

As used herein, the term “laminate” refers to a laminated product that includes at least one or two surfaces and a laminating material.

As used herein, the term “laminating material” refers to a material that can mate two surfaces or cover both sides of a single surface. For example, a laminating material may be a PVB substrate with a porous coating on top, or an adhesive material (which can include a porous coating) that allows the formation of a laminate.

As used herein, the term “oligomer” refers to a material that is soluble (e.g. water soluble) and has about 500 or less repeat units, or 200 or less, or 100 or less, or 50 or less, or 25 or less, or 10 or less.

As used herein, “partial thickness wound” refers to wounds that encompass Grades I-III; examples of partial thickness wounds include burn wounds, pressure sores, venous stasis ulcers, and diabetic ulcers. The term “deep wound” is meant to include both Grade III and Grade IV wounds. The present invention contemplates treating all wound types, including deep wounds and chronic wounds.

As used herein, reference to a “polyelectrolyte” intends a polymer material that contains or can be made to contain (e.g., by appropriately adjusting the pH of a solution containing the polyelectrolyte) a plurality of electrostatic charges. The term “polyelectrolyte” includes compounds or materials that contains multiple functional groups that maintain electrostatic interactions, dipole-dipole interactions or hydrogen bonding (e.g., alcohols, amines, sulfur-containing groups such as thionyl, polar groups such as carbonyls, and the like).

As used herein, “polymer multilayer” refers to the composition formed by sequential and repeated application of polymer(s) to form a multilayered structure. For example, hydrophilic polymer multilayers are polymer multilayers formed by the addition of polymers to a wound or support. The term “polymer multilayer” also refers to the composition formed by sequential and repeated application of polymer(s) to a wound or to a solid support. In addition, the term “polymer layer” can refer to a single layer composed of polymer molecules existing either as one layer within multiple layers on a wound or support. While the delivery of polymers to the wound bed or support can be sequential, the use of the term “polymer multilayer” is not limiting in terms of the resulting structure of the coating. It is well understood by those skilled in the art that inter-diffusion of polymers such as polyelectrolytes can take place leading to structures that may be well-mixed in terms of the distribution of the polymers used. It is also well understood by those skilled in the art that multilayer structures can be formed through a variety of interactions, including electrostatic interactions and others such as hydrogen bonding.

As used herein, the term “porous coating” refers to a porous coating covering a substrate, as well as any delamination products (e.g., films or particles) after a porous coating is removed from a substrate.

As used herein, “promote wound healing,” “enhance wound healing,” and the like refer to either the induction of the formation of granulation tissue of wound contraction and/or the induction of epithelialization (i.e., the generation of new cells in the epithelium). Wound healing is conveniently measured by decreasing wound area.

As used herein and unless otherwise specified, the term “solution” refers to a combination of at least one component in a liquid phase with at least one additional component dispersed or dissolved therein. The term includes homogeneous solutions (i.e., where the additional component is completely soluble in the liquid component). The term also includes mixtures (i.e., where the additional component is a solid that is not soluble or is not completely soluble in the liquid component).

As used herein, the term “sparingly soluble” refers to a material with a solubility of about 100 g/L or less, 50 g/L or less, 20 g/L or less, or 10 g/L or less, or 1 g/L or less, or 0.5 g/L or less, or 0.1 g/L or less.

As used herein and unless indicated otherwise, the term “substrate surface” (or sometimes simply “surface”), includes the surface of a substrate itself as well as the surface of any coatings deposited on the substrate (including a portion of a layer-by-layer coating), as well as a liquid layer present on a surface. Thus, for example, when a material is deposited on a substrate surface, the material may be deposited directly onto the surface of the substrate itself, or the material may be deposited onto the surface of a coating disposed on the substrate.

As used herein, “surfactant” refers to an amphiphilic material that modifies the surface and interface properties of liquids or solids. Surfactants can reduce the surface tension between two liquids. Detergents, wetting agents, emulsifying agents, dispersion agents, and foam inhibitors are all surfactants.

As used herein, the “thickness” of a bilayer refers to the average distance between the center of the nanoparticles that form the bilayer and the center of the nanoparticles that form an adjacent bilayer. With this definition, the following will be appreciated. First, the “center” of the nanoparticles of a given layer refers to a hypothetical plane intersecting the nanoparticles in such a way that minimizes the summation of the perpendicular distances between the plane and the center of each individual nanoparticle. Second, this definition is only relevant for a coating having more than one bilayer, and for a coating having “n” bilayers, only n−1 thicknesses are definable. Third, each bilayer having two adjacent bilayers (i.e. one above and one below) can have two thicknesses.

As used herein, by a “tightly packed” layer of nanoparticles is meant that the nanoparticles form a substantially homogeneous monolayer with a high packing density of nanoparticles. By high packing density, this includes packing arrangements that include hexagonal close packed, random close packed, and other close packings known in the art. In some embodiments the three-dimensional density of monodisperse nanoparticle is greater than 50%, or greater than 55% or greater than 60%. In some aspects the three dimensional density of monodisperse nanoparticle is between 50-64%, or 55-64, or 60-64%.

As used herein, “wound” refers broadly to injuries to the skin and subcutaneous tissue initiated in different ways (e.g., pressure sores from extended bed rest and wounds induced by trauma) and with varying characteristics. The methods and compositions described herein are useful for treatment of all types of wounds, including wounds to internal and external tissues, and wounds induced during medical procedures (e.g., surgical procedures) (e.g., abdominal surgery, hernia surgery, gastrointestinal surgery, bariatric surgery, reconstruction surgery, dural membrane surgery, etc.). Wounds may be classified into one of four grades depending on the depth of the wound: i) Grade I: wounds limited to the epithelium; ii) Grade II: wounds extending into the dermis; iii) Grade III: wounds extending into the subcutaneous tissue; and iv) Grade IV (or full-thickness wounds): wounds wherein bones are exposed (e.g., a bony pressure point such as the greater trochanter or the sacrum).

As used herein, “wound dressing” refers to materials placed proximal to a wound that have absorbent, adhesive, protective, osmoregulatory, pH-regulatory, or pressure-inducing properties. Wound dressings may be in direct or indirect contact with a wound. Wound dressings are not limited by size or shape. Indeed, many wound dressing materials may be cut or configured to conform to the dimensions of a wound. Examples of wound dressing materials include but are not limited to gauze, adhesive tape, bandages, and commercially available wound dressings including but not limited to adhesive bandages and pads from the Band-Aid® line of wound dressings, adhesive bandages and pads from the Nexcare® line of wound dressings, adhesive bandages and non-adhesive pads from the Kendall Curity Tefla® line of wound dressings, adhesive bandages and pads from the Tegaderm® line of wound dressings, adhesive bandages and pads from the Steri-Strip® line of wound dressings, the COMFEEL® line of wound dressings, adhesive bandages and pads, the Duoderm® line of wound dressings, adhesive bandages and pads, the TEGADERM™ line of wound dressings, adhesive bandages and pads, the OPSITE® line of wound dressings, adhesive bandages and pads, and biologic wound dressings. A “biologic wound dressing” is a type of wound dressing that comprises, e.g., is coated with or incorporates, cells and/or one or more biomolecules or fragments of biomolecules that can be placed in contact with the wound surface. The biomolecules may be provided in the form of an artificial tissue matrix. Examples of such biomolecules include, but are not limited, to collagen, hyaluronic acid, glycosaminoglycans, laminin, vitronectin, fibronectin, keratin, antimicrobial polypeptides and combinations thereof. Examples of suitable biologic wound dressings include, but are not limited to, BIOBRANE™, Integra™, Apligraf®, Dermagraft®, Oasis®, Transcyte®, Cryoskin® and Myskin®.

### Polymers

For wound care applications, the polymer can be water soluble and bio-resorbable (biodegradable and biocompatible), for example polyvinyl alcohol (PVA), polycaprolactone (PCL), polylactic acid (PLA), poly(lactic-co-glycolic acid) (PLGA), polyurethane (PU), and polyethylene oxide/polyethylene glycol (PEO/PEG), polyvinylpyrrolidone (PVP).

A dressing for a wound can be biocompatible, acting as a physical barrier against microorganisms while allowing gas permeation to keep the wound hydrated and remove excess exudate. Additionally, desirable properties include good mechanical strength and flexibility. Non-toxicity, biocompatibility, and biodegradability are also important criteria for materials used in dressings. Hydrogels, polymer films, foams, gauzes, and hydrocolloids are among the most extensively used dressings, depending on the wound type and therapeutic needs.

The thin films described herein can be applied to a wound, a biologic tissue, a cornea, a lens, a bone, a tendon, a surgical mesh, a wound dressing, a biomedical device, a device used for healthcare, or other surface. The thin film can be functionalized. The thin film can have one or more polymers, preferably biocompatible, or is formed from one or more proteins, or is a combination of polymers and proteins. The thin film can be made of synthetic polymers such as synthetic polyelectrolytes. The thin can be made from naturally occurring polymers such as polysaccharides. A peroxide and/or other additives, for example, an antimicrobial agent such as silver, polyhexamethylene biguanide (PHMB), chlorhexidine, or iodine compound, or an antibiotic, is incorporated into the thin film. The peroxide and/or other additives can be impregnated, incorporated or interspersed throughout the three dimensional structure of the thin film. The thin film can be made of multiple layers of the same or different hydrophilic polymers.

Thin films described herein can be made from hydrophilic polymers such as polyvinyl alcohol (PVA), polydiallyldimethylammonium chloride, polyacrylic acid, sulfonated polystyrene, chitosan, chitin, carboxymethylcellulose, hyaluronic acid, polyvinylpyrollidone, polyvinylalcohol, polyallylamine, polythiophenes, polyethyleneimines, polyacrylamides or copolymers or combinations thereof. In an aspect, the thin film is made from a hydrophilic polymer that is GRAS (generally regarded as safe) including, for example, polysaccharides, polyvinyl alcohol (PVA), chitosan, starch, alginate, dextran, dextrin, chitin, guar gum, gum karaya, agar, Fenugreek seed mucilage, Soy polysaccharide, Gellan gum, Mango peel pectin, Lepidium sativum mucilage, Plantago ovata seed mucilage, Aegle marmelos gum, Locust bean gum, Lepidium sativum, Mangifera indica gum, Hibiscus rosa-sinensis mucilage, carrageenan, hyaluronic acid, carboxymethylcellulose, carnauba wax, carob bean gum, carotene, cellulose, gelatin, gum Arabic, gum Ghatti, gum gualac, gum tragacanth, hydroxypropylmethyl cellulose, polyethylene glycol (PEG), methylcellulose, polyethylene glycol (PEG), propylene glycol.

Thin films described herein can also be made from other hydrophilic polymers such as, for example, polyacrylamide, poly(2-acrylamido-2-mewthylpropane sulfonic acid), poly(2-acrylamido-2-methylpropanesulfonic acid), poly(N,N-diethyl acrylamide), ploy (N-isopropyl acrylamide), poly(N,N-dimethyl acrylamide), poly(N,N-dimethylaminopropyl acrylamide), poly(N-phenethyl methylacrylamide), poly(acrylic acid), poly(alpha-ethylacrylic acid), poly(methacrylic acid), poly(alpha-propylacrylic acid), poly(2-aminoethyl methacrylate), poly(2-hydroxyethyl methacrylate), poly(N,N-dimethylaminoethyl methacrylate), poly(4-styrene sulfonic acid), poly(N-vinyl acetamide), poly(N-vinyl formamide), poly(N-vinyl isobutyramide), poly(vinylamide), poly(N-vinyl pyrrolidone) (PVP polymers), poly(2-vinyl pyrazine), poly(N-vinyl imidazole), poly(2-vinyl pyridine), poly(ethylene imine), poly(methyl vinyl ether), poly(oxymethylene), poly(tetrahydrofuran), polyglutamic acid, acrylic polymers and copolymers include acrylic acid, acrylamide, and maleic anhydride polymers and copolymers, for example alylamines, ethylenimines, oxazolines, and other polymers with amine groups in their main or side chains are examples of amine-functional polymers, ether polymers, fluorocarbon polymers, polystyrene polymers, poly(vinylchloride) polymers, natural polymers, semisynthetic polymers, and synthetic polymers. For example, see the polymers in Erothu et al, Hydrophilic Polymers (ed. Raju Francis and Sakthi Kumar), 2016, Wiley doi.org/10.1002/9783527690916.ch7, which is incorporated by reference in its entirety for all purposes.

The hydrophilic polymers can be positively charged or negatively charged. Examples of positively charged polymers include, for example, poly(allylamine hydrochloride) (PAH), polyl-lysine (PLL), poly(ethylene imine) (PEI), poly(histidine), poly(N,N-dimethyl aminoacrylate), poly(N,N,N-trimethylaminoacrylate chloride), poly(methyacrylamidopropyltrimethyl ammonium chloride), and natural or synthetic polysaccharides such as chitosan. Examples of negatively charged polymers include, for example, poly(acrylic acid) (PAA), poly(styrenesulfonate) (PSS), alginate, hyaluronic acid, heparin, heparan sulfate, chondroitin sulfate, dextran sulfate, poly(methacrylic acid), oxidized cellulose, carboxymethyl cellulose, polyaspartic acid, and polyglutamic acid.

The thin films can also incorporate amphoteric polymers, alone in combination with the other polymers described herein. Amphoteric polymers include, for example, one or more of acrylic acid (AA), DMAEMA (dimethylaminoethyl methacrylate), APA (2-aminopropyl acrylate), MorphEMA (morpholinoethyl methacrylate), DEAEMA (diethylaminoethyl methacrylate), t-ButylAEMA (t-butylaminoethyl methacrylate), PipEMA (piperidinoethyl methacrylate), AEMA (aminoethyl methacrylate), HEMA (2-hydroxyethyl methacrylate), MA (methyl acrylate), MAA (methacrylic acid) APMA (2-aminopropyl methacrylate), AEA (aminoethyl acrylate). The amphoteric polymer can include (a) carboxylic acid, (b) primary amine, and (c) secondary and/or tertiary amine. The amphoteric polymers can have an isoelectric point of 4 to 8, preferably 5 to 7 and have a number average molecular weight in the range of 10,000 to 150,000.

Other polymers that can be used to make the thin films are described in U.S. patent application Ser. No. 18/155,518, filed Jan. 17, 2023, (publication no. US20240009342) which is incorporated by reference in its entirety for all purposes.

The molecular weight of the hydrophilic polymer can be from 1-10000 kDa, 100 to 10000 kDa, 500 to 10000 kDa, 1000 to 10000 kDa, 50 to 500 kDa or 500 to 5000 kDa. The hydrophilic polymer can have a multimodal molecular weight distribution in the range 1 to 10000 kDa, 100 to 10000 kDa, 500 to 10000 kDa, 1000 to 10000 kDa, 50 to 500 kDa or 500 to 5000 kDa or can be a mixture of multiple polymers of unimodal or multimodal molecular weight distribution in the range 1-10000 kDa, 100 to 10000 kDa, 500 to 10000 kDa, 1000 to 10000 kDa, 50 to 500 kDa or 500 to 5000 kDa. The concentration of hydrophilic polymer in aqueous solution can be 1 to 10000 mM, 10 to 10000 mM, 100 to 10000 mM, 10 to 1000 mM, 10 to 500 mM, 10 to 50 mM, 1 to 50 mM, or 1 to 100 mM based on polymer repeat unit. The pH of the aqueous solution can be adjusted so that the hydrophilic polymer is at least 0.01% charged. The concentration of inorganic or organic salts can be from 1 to 10000 mM, 10 to 10000 mM, 100 to 10000 mM, 10 to 1000 mM, 10 to 500 mM, 10 to 50 mM, 1 to 50 mM, or 1 to 100 mM in the aqueous solution.

The thin films herein can have a surface area of at least 0.65, 1, 2, 5, 10, 100 or 500 square meters or from 0.65 to 1.0, 0.65 to 5.0, 0.65 to 10, 0.65 to 20, 0.65 to 50, 0.65 to 100, 0.65 to 200, 0.65 to 300, 0.65 to 400, 0.65 to 500, 1 to 10, 1 to 20, 1 to 50, 1 to 100, 1 to 200, 1 to 300, 1 to 400, 1 to 500, 2 to 10, 2 to 20, 2 to 50, 2 to 100, 2 to 200, 2 to 300, 2 to 400, 2 to 500, 5 to 10, 5 to 20, 5 to 50, 5 to 100, 5 to 200, 5 to 300, 5 to 400, 5 to 500, 10 to 20, 10 to 50, 10 to 100, 10 to 200, 10 to 300, 10 to 400, 10 to 500, 20 to 50, 20 to 100, 20 to 200, 20 to 300, 20 to 400, 20 to 500, 50 to 100, 50 to 200, 50 to 300, 50 to 400, or 50 to 500 square meters

### Peroxides and Other Additives

The thin films described herein can function as a payload delivery scaffold to deliver one or more peroxide and/or other additive to a desired sight. Other additives can include, for example, trophic factors, extracellular matrices (ECMs), ECM fragments or synthetic constructs, enzymes, enzyme inhibitors, defensins, polypeptides, anti-infective agents (including antimicrobials, antivirals and antifungals), buffering agents, vitamins and minerals, analgesics, anticoagulants, coagulation factors, anti-inflammatory agents, vasoconstrictors, vasodilators, diuretics, and anti-cancer agents. Other additives can also include chlorhexidine, iodine based antimicrobials such as PVP-iodine; selenium based antimicrobials such as 7-azabenzisoselenazol-3 (2H)-ones, selenium disulfide, and selenides; silver based antimicrobials (e.g., silver sulfadiazine, ionic silver, elemental silver, silver nanoparticles)) and gallium based antimicrobials. Using standard and variations of typical protein and carbohydrate attachment chemistries, carboxyl and amino containing selenides may be routinely attached to many polymers, peptides, antibodies, steroids and drugs. Polymers and other molecules with attached selenides can generate superoxide in a dose dependent manner. Additives that can be included in the thin films are described as bioactive agents in U.S. patent application Ser. No. 18/155,518, filed Jan. 17, 2023, (publication no. US20240009342) which is incorporated by reference in its entirety for all purposes.

The thin films can contain agents that kill microorganisms or inhibit the growth of microorganisms. Such agents include, for example, antiseptics, antibiotics, antivirals, antifungals, antimicrobials, and antiparasitics. Antimicrobials can kill microorganisms and/or prevent their growth by targeting key steps in cellular metabolism such as the synthesis of biological macromolecules, the activity of cellular enzymes, or cellular structures such as the cell wall, cell membranes.

Peroxide and/or peroxide generating agents can be added to the thin films. For example, the thin film can include hydrogen peroxide, calcium peroxide, sodium peroxide, lithium peroxide, barium peroxide, magnesium peroxide, zinc peroxide, carbamide peroxide, benzoyl peroxide (e.g., for acne treatment), and hydrogen peroxide-urea. The peroxide agent can be included in the thin film and make up 1-30% by weight of the thin film. In an aspect, the amount of peroxide in the thin film can be 3-25% by weight, 3-10% by weight, 3-5% by weight, 5-20% by weight, 5-15% by weight, 5-10% by weight, 10-30% by weight, 10-20% by weight and 10-15% by weight. The amount of peroxide in the thin film by weight can be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25%.

Other additives useful for wound care and/or skin care can be included in the thin films such as, for example, antibiotics, salicylic acid, hydroquinone, retinoids, hyaluronic acid, and/or vitamin C. Antibiotics can include, for example, aminoglycosides (e.g., gentamicin, amikacin, tobramycin, neomycin, and streptomycin), sulfonamides (e.g., Mafenide, Sulfacetamide, Sulfadiazine, Sulfadoxine, Sulfamethizole, Sulfamethoxazole, Sulfanilamide, Sulfasalazine), tetracyclines (e.g., lymecycline, methacycline, minocycline, rolitetracycline, and doxycycline), carbapenems (e.g., imipenem-cilastatin, meropenem, ertapenem, doripenem, panipenem-betamipron, and biapenem), cephalosporins (e.g., cefazolin, cephalexin, cefuroxime, cefoxitin, ceftriaxone, ceftazidime, cefepime, and ceftaroline), 2- and 4-quinolones (e.g., nalidixic acid, 6-fluoroquinolone, ciprofloxacin, ofloxacin, levofloxacin, moxifloxacin, balofloxacin, grepafloxacin, pazufloxacin, sparfloxacin, temafloxacin, gatifloxacin, and trovafloxacin), glycopeptides (e.g., vancomycin, teicoplanin, ramoplanin, oritavancin, dalbavancin, and telavancin), penicillin (e.g., amoxicillin, amoxicillin/clavulanic acid, ampicillin, benzylpenicillin, benzathine benzylpenicillin, dicloxacillin, flucloxacillin, and phenoxymethylpenicillin (Penicillin V)), rifamycin (e.g., rifampicin, rifabutin, rifapentine, and rifaximin), monobactams (e.g., tigemonam, nocardicin A, tabtoxin, azactam, aztreonam), oxazolidinone antibiotics (e.g., Linezolid, Sivextro, Tedizolid, and Zyvox), streptogramins (e.g., Quinupristin, pristinamycin, and virginiamycin), and polypeptide antibiotics (e.g., actinomycin, bacitracin, colistin, and polymyxin B).

Other additives can also include antimicrobial agents including, for example, metallic particles, and metal ion antimicrobial agents. The metal ion antimicrobial agent can be a metal ion, metal ion salt, or metal ion nanoparticle. The metal ion nanoparticle can be a silver nanoparticle. The antimicrobial agent can be, for example, silver, chlorhexidine, antibiotics, polyhexamethylene biguanide (PHMB), iodine, cadexomer iodine, povidone iodine (PVI), hydrogen peroxide, and vinegar (acetic acid). The additive can be an antibiofilm agent such as, for example, small molecule antibiofilm agents, charged small molecule antibiofilm agents, antibiofilm polypeptides, antibiofilm enzymes, metallic particles, and metal ion antibiofilm agents. A metal ion antibiofilm agent can be a metal ion, metal ion salt, or metal ion nanoparticle. The metal ion antibiofilm agent can be a gallium ion, gallium ion salt, gallium ion nanoparticle, gallium alloy, or an alloy of gallium and silver. The antibiofilm enzyme can be Dispersin B.

The additive can also be a growth factor, a hemostatic agent, a bioactive peptide, a bioactive polypeptide, an analgesic, an anticoagulant, an anti-inflammatory agent, and a drug molecule or a drug compound.

The peroxide and/or other additive can be applied to form a gradient in the thin film. In general, the gradients present a higher contraction of peroxide and/or other additive at one or more first desired locations and a lower concentration of peroxide and/or other additive at one or second location. For example, the concentrations of the peroxide and/or other additive can be layered in a thin film in a gradient such that higher concentrations are proximal to the application site (e.g., wound) than distal to the application site (e.g., wound) in a vertical fashion. The converse, where concentrations of compositions is greater distal to the application site (e.g., wound) than proximal, is also contemplated. Concentration of compositions in a application site (e.g., wound) wherein a horizontal gradient is deposited is also contemplated. Topographical gradients are also contemplated, wherein compositions are deposited such that the concentrations of compositions in an application site (e.g., wound) or on a biocompatible particle follow the topography of the substrate, for example, a higher concentration of compositions can be deposited in the valleys of undulations of an exemplary substrate compared to the peaks of the undulations.

The gradient can provide a higher concentration of the peroxide and/or other additive in the center of the application site which transitions to a lower concentration of the peroxide and/or other additive away from the center of the application site. Accordingly, when the thin film is applied to a site, the gradient results in a higher concentration of peroxide and/or other additive in the center of the application site and a lower concentration of peroxide and/or other additive as one moves to the periphery of the application site. The gradient can provide a lower concentration of the peroxide and/or other additive in the center of the application site which transitions to a higher concentration of the peroxide and/or other additive away from the center of the application site. Accordingly, the gradient results in a lower concentration of peroxide and/or other additive in the center of the application site and a higher concentration of peroxide and/or other additive as one moves to the periphery of the application site. If two or more peroxide and/or other additive are utilized, they can be presented as similar gradients or the gradients can be varied so that the concentrations of the two or more peroxide and/or other additive vary across the application site. The gradients of high or low concentration can be any shape, such as circular, square, rectangular, oval, oblong, etc. so that the matrix and gradient can conform to a variety of wound shapes. For example, for long, incision type wounds, the gradient may be centered on a longitudinal axis that extends along the length of the wound and can be centered on the wound. As another example, the gradient can be circular or oval-shaped for application to open type wounds, burns, sores and ulcers that are roughly circular or oval. In other embodiments, the gradients comprise a series of features arranged in a pattern. For example, the gradients can form a series of stripes or high and low concentrations of one or more bioactive agents along a longitudinal axis of the matrix. Alternatively, the gradients can form a checkerboard pattern, array, concentric circles, overlapping circles or oval, etc.

### Methods for Making Thin Films

Disclosed herein are processes for manufacture of a thin film comprising: a) providing a flexible substrate and having a surface area of greater than 0.52 square meters; b) depositing a polymer layer the low substrate surface; and c) introducing a bioactive agent into the polymer layer to provide a bioactive polymer layer. In an aspect, the surface area of thin film is greater than 0.65 square meters.

Thin films of the disclosure can be made by making a layer or layers of polymer (dissolved in water) applied using the following wet coating techniques, after which the wet film has to be dried to remove the solvent by convection using hot air, to speed things up, or ambient air. Applicable wet coating techniques include, for example, reverse-roll coating, knife-over-roll coating, Meyer rod coating, gravure, slot-die, dip coating, spin coating, and spray coating. Other methods for making the thin films herein include, for example, immersion, inkjet, flexographic, metering rod, blade, air knife, curtain, melt extrusion, solvent casting and any combinations of the methods described above. See, e.g., US PAT PUBL. 20140079884, US PAT PUBL. 20160068703, US PAT PUBL. 20120269973, US PAT PUBL. 20160114294, US PAT PUBL. 20140112994, US PAT PUBL. 20150086599, Shiratori, Japanese Journal of Applied Physics Vol. 44, No. 3, 2005, L126-L128, and Grunlan, Industrial & Engineering Chemistry Research Vol. 53, 2014, 6409-6416, all of which are incorporated herein by reference in their entirety.

Reverse-roll coating utilizes a roller in close proximity to a moving web, which roller spins in the reverse direction of the plastic, forming a bead of fluid that travels with the passing plastic based on the gap between roller and plastic, and the viscosity of the solution. Reverse roll coating is described in, for example, Alonso et al., Process viscosity in reverse roll coating, Chemical Engineer. Res. Design 79:128-136 (2001); Ostness, Coating technology for flexible packaging, tappi.org/content/enewsletters/eplace/2006/06PLA50.pdf; Kapur, Flow phenomena in fixed-gap and gravure roll coating systems, etheses.whiterose.ac.uk/929/1/uk_bl_ethos_366501.pdf; ali et al., Theoretical study of the reverse roll coating of non-isothermal magnetohydrodynamics viscoplastic fluid, Coatings 10:940 (2020), each of which is incorporated by reference in its entirety for all purposes. Reverse-roll coating processes can be performed on commercially sold machines including, for example, those sold by New Era Converting Machinery Inc., Xiamen Simy Equipment Limited Co., Ltd., Dubois Equipment Company, LLC, Schaefer Machine Co., Pyradia Belfab, Technical Coating International, and the Union Tool Corporation.

Knife-over-roll coating uses a tapered blade positioned slightly above a passing web of plastic, allowing only a thin layer of fluid to remain on the plastic as it passes underneath. Knife over roll coating is described in, for example, Herrera, Versatility in coating operations—knife coatings, Coated Fabrics. 1991;20(4):289-301. doi:10.1177/152808379102000408; Coyle, D. J. (1997). Knife and Roll Coating. In: Kistler, S. F., Schweizer, P. M. (eds) Liquid Film Coating. Springer, Dordrecht. doi.org/10.1007/978-94-011-5342-3_15; Grant, Application of Coatings to Continuous Webs. Journal of Coated Fabrics. 1977;7(1):43-57, doi:10.1177/009346587700700104, each of which is incorporated by reference in its entirety for all purposes. Knife-over roll coating processes can be performed on commercially sold machines including, for example, those sold by Jessup Manufacturing Company, Delpro Limited, US Web Converting Machinery Corporation, National Coating Corporation, and Technical Coating International.

Meyer rod coating uses a rod with a spiral wrapped wire positioned such that it barely touches a passing plastic surface. The wire touches the film surface, and the rod in the middle is separated from the surface by the wire diameter. Only fluid which can fit through the gaps in the wire voids is allowed to remain on the passing plastic. Once it passes the Meyer rod the peaks and troughs created by the wire smooth out and level themselves prior to curing. Meyer rod coating is described in Kim et al, Multi-purpose overcoating layers based on PVA/silane hybrid composites for highly transparent, flexible, and durable AgNW/PEDOT:PSS films, RSC Adv. 6:19280-19287 (2016); Li et al., Roll-to-roll fabricating MXene membranes with ordered interlayer distanced for molecule and ion separation, Adv. Mater. Interfaces vol. 10, DOI:10.1002/admi.202300301 (2023), each of which is incorporated by reference in its entirety for all purposes. Meyer rod coating processes can be performed on commercially sold machines including, for example, those sold by Conversion Technologies, Inc., and Xiamen Simy Equipment Limited Co., Ltd.

Gravure uses a dimpled roller that dips into a trough of fluid and carries only what it can hold by surface tension out of the pan, a doctor blade scrapes the surface of the pits such that only fluid contained in the volume of the pits is allowed to remain. This dimpled roller then presses against a passing plastic surface, which is pressed into the dimples sufficiently to draw out all the fluid. These tiny mounds of fluid then settle into a liquid sheet of prescribed thickness before drying. Gravure coating is described in, for example, Wang et al., Large-area Gravure-printed AgNWs electrode on water/oxygen barrier substrate for long-term stable large-area flexible organic solar cells, Chinese J. Chem. 42:478-484 (2023); Kopola et al., High efficient plastic solar cells fabricated with a high-throughput gravure printing method, Solar Energy Mat. Solar Cells 94:1673-1680 (2010); Sato et al., Stretchable, adhesive and ultra-conformable elastomer thin films, Soft Matter 12:9202-9209 (2016), each of which is incorporated by reference in its entirety for all purposes. Gravure coating processes can be performed on commercially sold machines including, for example, New Era Converting Machinery, Inc., Retroflex, Inc., Pyradia Belfab, Hirano Tecseed Co., Ltd., and Kerone Engineering Solutions, Inc.

Slot-die uses fluid that is metered onto a passing plastic surface by pressure through a narrow precisely machined slot between two metal surfaces. This technique requires very precise machining and a defect free gap between the two. Slot-die is described in, for example, Parsekian et al., Scalable, alternating narrow stripes of polyvinyl alcohol support and unmodified PEDOT: PSS with maintained conductivity using a single-step slot die coating approach, ACS Appl. Mater. Interfaces 12:3736-3745 (2020); Jeong et al., Scaled production of functionally gradient thin films using slot die coating on a roll-to-roll system, ACS Appl. Mater. Interfaces 16:9264-9274 (2024); Lin et al., Minimum wet thickness for double-layer slide-slot coating of poly(vinyl-alcohol) solutions, Polymer Engineer. Sci. 45:1590-1599 (2005), each of which is incorporated by reference in its entirety for all purposes. Slot-die processes can be performed on commercially sold machines including, for example, Ossila, FOM Technologies, MTI Corporation, MIRWEC Coating, and nTact.

In dip coating a passing plastic surface is submerged into a pan of fluid as it rides around a roller face. Anything that carries along and doesn't drain back into the pan is dried into a sheet of film. This technique is only applicable for specific viscosity ranges and speeds. In dip coating is described in, for example, Schiessl et al., Nanocomposite coatings based on polyvinyl alcohol and montmorillonite for high-barrier food packaging, Sec. Nutrition Food Sci. Technol. Vol. 9, doi.org/10.3389/fnut.2022.790157 (2022); Sinturel et al., Influence of PLGA nanoparticles on the deposition of model water-soluble biocompatible polymers by dip coating, Colloids and Surfaces A: Physiochemical and Engineering Aspects, 608:125591 (2021), each of which is incorporated by reference in its entirety for all purposes. In dip coating processes can be performed on commercially sold machines including, for example, Xiamen Tmax Battery Equipment Limited., Ossila, Specialty Coating Systems, Inc., MSE Supplies, LLC, and PAR Systems.

Spin coating uses a fluid applied to the middle of a spinning substrate. As viscous forces overcome inertial forces, the fluid coats the surface as a thin layer. This technique is limited in size of substrate to that which can be spun safely. Spin coating is described in, for example, Manikandan et al., Construction of spin coating machine controlled by arm processor for physical studies of PVA, Intl J. Electronics Electrical Engineer. 3:318-322 (2015); Augustine et al., Excellent UV absorption in spin-coated thin films of oleic acid modified zinc oxide nanorods embedded in Polyvinyl alcohol, J. Phys. Chem. Solids 73:396-401 (2012); Moreira et al., Spin-coated freestanding films for biomedical applications, J. Mat. Chem. B, 9:3778-3799 (2021), each of which is incorporated by reference in its entirety for all purposes. Spin coating processes can be performed on commercially sold machines including, for example, Ossila, Specialty Coating Systems, Inc., Holmarc Opto-Mechatronics Ltd., MTI Corporation, and MicroNano Tools.

Spray coating sprays fluid onto a surface but uniformity is challenging or very expensive equipment is required. Spray coating is described in, for example, Wei et al., Constructing anti-scaling and anti-wetting polyvinyl alcohol layers through spray-coating with improved water permeability in membrane distillation, Desalination, Volume 545, article id. 116161 (2023); Wang et al., Spray-coated tough thin film composite membrane for pervaporation desalination, Chem. Engineer. Res. Design 179:493-501 (2022), each of which is incorporated by reference in its entirety for all purposes. Spray coating processes can be performed on commercially sold machines including, for example, SonoTek Corporation, SPS International, MTI Corporation, and Holmarc Opto-Mechatronics Ltd.

The thin films herein can be made on a substrate. The substrate can be a flexible sheet. The flexible sheet can be amenable to storage and winding onto a roll for use in a continuous roll-to-roll process. The flexible sheet can include, for example, a polyester film, a polyethylene terephthalate (PET) film, a biaxially oriented PET film, a polycarbonate, a polyethylene (including high density polyethylene, medium density polyethylene, low density polyethylene, linear low density polyethylene) film, a polyvinyl chloride film, a polyvinylidene chloride film, a polyvinylidene fluoride film, a nylon film, a polystyrene film, an acetate film, a polyurethane film, an ethylene vinyl acetate copolymer film, a cast polypropylene film, an uniaxially oriented polypropylene film and a biaxially oriented polypropylene films. The flexible sheet can include a release coating which can be a silicone release film, a polydimethyl siloxane (PDMS) coating, a fluorocarbon coating, a polyacrylate coating, a polystyrene coating, a polystyreneacrylic coating, a chromium sterate complex coating, or a polyolefin coating. Suitable release films include, but are not limited, to those provided St. Gobain Performance Plastics, Worcester Mass., such as Saint Gobain 4130, 4159 and 7819 release coatings. The release coating can allow complete peeling of the thin film coating without tears or other defects thus resulting in a free standing hydrophilic polymer film which can incorporate peroxide and/or other additives as described above.

Other materials may be substituted for the flexible sheet. Such other materials may be a paper or cellulosic substrate such as glassine or supercalendered kraft paper coated with a release coating.

The processes for making the thin films can also include steps to control the amount of the peroxide and/or other additives in the hydrophilic polymer thin film by controlling the number of polymer layers, by controlling the pH when forming the polymer layer, and/or by controlling the number of introducing cycles. The processes can control the amount of the peroxide and/or other additives in the polymer thin film by controlling the amount of peroxide and/or other additive introduced to the a polymer layer, controlling the concentration of peroxide and/or other additives, and/or by controlling the residence time of polymer layer in a solution with the peroxide and/or other additives.

The processes can form or deposit a second polymer layer (and/or multiple polymer layers) on the hydrophilic polymer-layer. The second polymer layer can slow the release of peroxide and/or other additives from nanoscale layer by 1 to 1000 times, 1 to 100 times, 10 to 1000 times, 20 to 1000 times, 50 to 1000 times, 100 to 1000 times, 10 to 500 times, 50 to 500 times, 10 to 200 times or 20 to 200 times.

The second polymer layer can be a sacrificial polymer layer. The sacrificial polymer layer can be dissolvable or biodegradable and be made of a water soluble polymer. The water soluble polymer can be polyvinyl alcohol (PVA). The water soluble polymer can have a molecular weight of less than 23 kDa. The water soluble polymer can have a molecular weight such that it can be removed from the blood by renal filtration. The sacrificial polymer layer can have a water soluble polymer dissolves when exposed to moisture on a surface so that the bioactive nanoscale polymer layer is deposited on the surface. In some embodiments, the second polymer layer comprises polyacrylic acid (PAA), polyvinylpyrrolidone (PVP), carboxymethyl cellulose (CMC), hydroxypropyl cellulose, hydroxypropyl methylcellulose, methylcellulose, ethylcellulose, hydroxyethyl cellulose (HEC), alginates, polyvinylacetate (PVAc), polylactic acid (PLA), polylactic-co-glycolic acid (PLGA), polyglycolic acid, or polyanhydrides.

The second polymer layer can include additives as discussed above, or can include surfactants, emulsifiers, wetting agents, rheology modifiers, plasticizers, emollients, humectants, disintegrants, lubricants, binders, compatibilizing agents, antistatic agents, and fillers. The second polymer layer can be from about 0.1 μm thick to about 100 μm thick, from about 0.1 μm thick to about 50 μm thick, from about 1 μm thick to about 20 μm thick, or from about 1 μm thick to about 10 μm thick.

The secondary polymer film can have a thickness of from 1-50 μm, preferably 5-25 μm, and can be coated onto the hydrophilic polymer layer. The second polymer layer can be made of a hydrogel, a hydrocolloid, and/or a collagen and such a second layer can provide support to the hydrophilic polymer layer.

An adhesive material can also be added to the thin films herein. Adhesive materials include, for example, polyvinyl butyral (PVB), polyacrylates, polymethyl methacrylates, vinyl acetates, polyvinyl alcohol, and the like. The adhesive material can be added to the thin film by any methods known in the art.

A freestanding sheet can be obtained by peeling the combined hydrophilic polymer layer and second polymer layer from the substrate. This freestanding film may be referred to as a microsheet. The substrate supporting the hydrophilic polymer layer and second polymer layer can be cut to a predetermined size and/or state before the microsheet is peeled away. The hydrophilic polymer layer of a microsheet can contain peroxide and/or other additives such as antimicrobial agents, antibiofilm agents, microparticles, nanoparticles, magnetic particles as described above. The microparticles or nanoparticles in the hydrophilic polymer layer can contain bioactive agents or antimicrobial agents. The second polymer layer can include an antibiofilm agent. For example, the antibiofilm agent can be a small molecule antibiofilm agent, a charged small molecule antibiofilm agent, an antibiofilm polypeptide, an antibiofilm enzyme (e.g., Dispersin B), a metallic particle, or a metal ion antibiofilm agent (e.g., a metal ion, metal ion salt, or metal ion nanoparticle). Further, the metal ion antibiofilm agent can be a gallium ion, a gallium ion salt, a gallium ion nanoparticle, an alloy of gallium, or an alloy of gallium and silver.

Peroxide and/or other additives can be incorporated into the hydrophilic polymer layer or second polymer layer, referred to collectively as a thin film and/or microsheet. The description herein is not limited to a particular mechanism by which the peroxide and/or other additives are released from the polymer layer(s). The mechanism by which release is achieved is not necessary to practice the embodiments described herein. Exemplary methods for release include, release of the one or more incorporated agents from the thin film by diffusion from the polymer layer. The peroxide and/or other additive may be released from the polymer layer(s) over time or in response to an environmental condition. The peroxide and/or other additive may be attached by a degradable linkage in a polymer layer, such as a linkage susceptible to degradation via hydrolysis or enzymatic degradation. The linkage may be one that is susceptible to degradation at a certain pH, for example.

The methods described herein can add a hydrogen peroxide-urea adduct to the thin film (e.g., PVA sheet). By mixing a solid adduct of hydrogen peroxide and urea, often called carbamide peroxide into the hydrophilic polymer solution (e.g., PVA) immediately prior to coating, the resulting thin film sheet contains stable hydrogen peroxide which, when placed on an open wound, produces an oxidative environment rich in reactive oxygen species (ROS) that are desirable for wound healing. The hydrogen peroxide can also react with catalase in the wound to produce a burst of oxygen and water, cleaning the wound and further providing ROS to promote wound healing. Carbamide peroxide also provides urea. Urea in low doses is a humectant, maintaining a moist environment in the vicinity of the wound, which is also applied to treat dry, itching skin such as may occur in psoriasis, dermatitis or ichthyosis, as well as to soften and promote healing of skin areas affected by hyperkeratosis. It can also be used to loosen and debride devitalized tissue in a wound bed that can act as a reservoir for bacterial growth.

Thin films made according to the methods herein described can have 25% hydrogen peroxide by weight, 41% urea by weight and 33% hydrophilic polymer (e.g., PVA) by weight. The final dry hydrophilic polymer film can have 5-25% hydrogen peroxide by weight, 8-41% urea by weight, and 33-86% hydrophilic polymer (e.g., PVA) by weight. The final dry hydrophilic polymer film can have 3-25% hydrogen peroxide by weight, 5-41% urea by weight, and 33-91% hydrophilic polymer (e.g., PVA) by weight. The final dry hydrophilic polymer film can have 10-25% hydrogen peroxide by weight, 16-41% urea by weight, and 33-73% hydrophilic polymer (e.g., PVA) by weight. The final dry hydrophilic polymer film can have 15-25% hydrogen peroxide by weight, 25-41% urea by weight, and 33-59% hydrophilic polymer (e.g., PVA) by weight. The final dry hydrophilic polymer film can have 20-25% hydrogen peroxide by weight, 33-41% urea by weight, and 33-46% hydrophilic polymer (e.g., PVA) by weight. The final dry hydrophilic polymer film can have 5-20% hydrogen peroxide by weight, 8-33% urea by weight, and 46-86% hydrophilic polymer (e.g., PVA) by weight. The final dry hydrophilic polymer film can have 3-20% hydrogen peroxide by weight, 5-33% urea by weight, and 46-91% hydrophilic polymer (e.g., PVA) by weight. The final dry hydrophilic polymer film can have 10-20% hydrogen peroxide by weight, 16-33% urea by weight, and 46-73% hydrophilic polymer (e.g., PVA) by weight. The final dry hydrophilic polymer film can have 15-20% hydrogen peroxide by weight, 25-33% urea by weight, and 46-59% hydrophilic polymer (e.g., PVA) by weight. The final dry hydrophilic polymer film can have 5-15% hydrogen peroxide by weight, 8-25% urea by weight, and 59-86% hydrophilic polymer (e.g., PVA) by weight. The final dry hydrophilic polymer film can have 10-15% hydrogen peroxide by weight, 16-25% urea by weight, and 59-73% hydrophilic polymer (e.g., PVA) by weight.

### Uses for Thin Films

The disclosure provides a freestanding thin film of a desired size and shape as described above, optionally including a peroxide and/or other additive (e.g., antimicrobial silver compounds, antimicrobial gallium compounds, or analgesic compounds). The thin film may have a desired size and shape by cutting the substrate material to a desired size and shape and peeling the thin film from the substrate. The thin film may then be applied to an application site such as a wound or a medical surface such as the surface of a medical device.

The thin film can be used to modify a wound dressing or biologic wound dressing that is compatible with functionalization by addition of a matrix material. Examples of commercially available wound dressings that can be modified by addition of a thin film include, but are not limited to, Biobrane™, gauze, adhesive tape, bandages such as Band-Aids®, and other commercially available wound dressings including but not limited to COMPEEL®, DUODERM™, TAGADERM™, and OPSITE®. In an aspect, the disclosure provides methods for transferring a thin film herein to a desired surface, such as soft surface. Such soft surfaces include, but are not limited to, skin, a wound bed, a tissue, artificial tissues including artificial skin tissues such as organotypically cultured skin tissues, Apligraf®, Dermagraft®, Oasis®, Transcyte®, Cryoskin® and Myskin®, artificial tissue matrices, gels comprising biomolecules, a wound dressing, and a biologic wound dressing. The desired surface can be contacted with a thin film herein, e.g., a polymer multilayer supported on a support and pressure is applied to effect transfer of the polymer multilayer from the support to the desired surface. The transfer can be performed in the substantial, or complete, absence of solution. The transfer can be performed through a gas phase. The transfer can be performed in an environment where the humidity is less than 100% of saturation. The transfer can be performed in the absence of liquid water.

In an aspect, the disclosure herein provides wound dressings with a support material having a surface oriented to a wound, wherein the surface oriented to the wound is modified with a thin film material described herein. When applied to a wound, the surface of the support material modified with the matrix material is put into contact with the wound bed.

The support can be a biologic wound dressing. Biologic wound dressings can be of the type having, e.g., a coating or incorporates, cells (e.g., keratinocytes or fibroblasts and combinations thereof) and/or one or more biomolecules or fragments of biomolecules that can be placed in contact with the wound surface. The biomolecules may be provided in the form of an artificial tissue matrix comprising one or more biomolecules. Examples of such biomolecules include, but are not limited, to collagen, glycosaminoglycans, hyaluronic acid, laminin, vitronectin, fibronectin, keratin, antimicrobial polypeptides and combinations thereof. Examples of suitable biologic wound dressings include, but are not limited to, BIOBRANE™, Integra™, Apligraf®, Dermagraft®, Oasis®, Transcyte®, Cryoskin® and Myskin®.

The thin film can be used to modify a biosynthetic wound dressing constructed of an elastomeric film supported on support material, such as a fabric, preferably a polymeric fabric such as a nylon fabric. The fabric can be at least partially imbedded into the film (e.g., BioBrane™). The elastomeric film can be coated with one or more biomaterials, for example collagen, keratin, fibronectin, vitronectin, laminin and combinations thereof. Accordingly, the fabric presents to the wound bed a complex 3-D structure to which a biomaterial (e.g., collagen) has been bound, preferably chemically bound. The surface presented to the wound can be further modified with a thin film as described above. The thin film can be hydrophilic polymer layer comprising a peroxide and/or other additive, e.g., selected from one or more of silver nanoparticles, elemental silver, and silver containing compounds such as silver sulfadiazine and/or gallium ions and related compounds, and preferably included in the concentration ranges described above.

The thin films can be used to modify an adhesive bandage comprising an adhesive portion (such as an adhesive strip) and an absorbent material, preferably treated or coated with a material (i.e., a non-adherent material) to prevent adhesion to the wound or comprising a layer of non-adherent material, such as Teflon®, on the surface of the absorbent pad that will contact the wound. The absorbent material can be an absorbent pad (e.g., a gauze pad or polymer foam) preferably treated or coated with a material (i.e., a non-adherent material) to prevent adhesion to the wound or having a layer of non-adherent material, such as Teflon® or other suitable material, on the surface of the absorbent pad that will contact the wound. The non-adhesive material or layer can be breathable. The bandage can also have a wound dressing made from a gel-forming agent, for example, a hydrocolloid such as sodium carboxymethylcellulose. The absorbent pads or gel-forming agents can be affixed to a material that is waterproof and/or breathable. Examples include, but are not limited, semipermeable polyurethane films. The waterproof and/or breathable material may further include an adhesive material for securing the bandage to the skin of a subject. The waterproof and/or breathable material preferably forms the outer surface of the adhesive bandage or pad, i.e., is the surface opposite of the surface comprising the matrix which contacts the wound. Examples of such adhesive bandages and absorbent pads include, but are not limited to, to adhesive bandages and pads from the Band-Aid® line of wound dressings, adhesive bandages and pads from the Nexcare® line of wound dressings, adhesive bandages and non-adhesive pads from the Kendall Curity Tefla® line of wound dressings, adhesive bandages and pads from the Tegaderm® line of wound dressings, adhesive bandages and pads from the Steri-Strip® line of wound dressings, the COMFEEL® line of wound dressings, adhesive bandages and pads, the Duoderm® line of wound dressings, adhesive bandages and pads, the TEGADERM™ line of wound dressings, adhesive bandages and pads, the OPSITE® line of wound dressings, adhesive bandages and pads, adhesive bandages and pads from the Allevyn line of wound dressings, adhesive bandages and pads from the Duoderm® line of wound dressings, and adhesive bandages and pads from the Xeroform® line of wound dressings.

A thin film as described herein can be applied to a wound under conditions such that wound healing, as measured by wound contraction, is accelerated. The thin film containing peroxide and/or other additives can be transferred to a wound or tissue so that a second polymer layer lies on top of the hydrophilic polymer layer after transfer to the wound or tissue. A wound dressing can be placed on top of thin film. Optionally, the second polymer layer can be removed before or after the wound dressing is applied. The thin film with peroxide and/or other additives can be transferred to a wound or tissue surface such that the thin film dissolves completely in the wound, and optionally a primary/secondary wound dressing can be placed over the wound. The primary/secondary dressing can be a biologic dressing and the thin film does not hinder integration of biologic dressing in the wound-bed.

Hydrogen peroxide can play a critical role in each of the four stages of wound healing (hemostasis, inflammation, proliferation, tissue remodeling). The peroxide concentrations can be different in each stage. For example, partial bacteriostatic effects of peroxide are observed at concentrations between 25-50 μM, while cell killing is observed at concentrations above 500 μM.

The thin films with peroxide and/or other additives can be provided as kits, preferably with the thin film in a sterile package. The thin film provided in the kit can have at least one peroxide and/or other additive. The kits can include a peroxide and/or other additive with instructions for applying the peroxide or other additive to the thin film prior to application to a desired site (e.g., skin or wound).

A thin film as described herein, can be applied to all types surfaces, e.g., wounds, skin, surface of a medical device. Furthermore, a peroxide and/or other additive can be applied to skin, mucous membranes, body cavities, and to internal surfaces of bones, tissues, etc. that have been damaged. A thin film with one or more peroxide and/or other additives can be used on wounds such as cuts, abrasions, ulcers, surgical incision sites, burns, and to treat other types of tissue damage or conditions. The thin films disclosed herein can enhance wound healing. Wound healing may be enhanced in a variety of ways by the thin films disclosed herein. The thin films disclosed herein and methods using the thin films can minimize contracture of the wound as to best favor function and cosmesis. The thin films described herein and methods using the thin films can promote wound contracture to best favor function and cosmesis. The thin films described herein and methods using the thin films can promote vascularization. The thin films described herein and methods using the thin films can inhibit vascularization. The thin films described herein and methods using the thin films can promote fibrosis. The thin films described herein and methods using the thin films can inhibit fibrosis. The thin films described herein and methods using the thin films can promote epithelial coverage. The thin films described herein and methods using the thin films can inhibit epithelial coverage. The thin films described herein and methods using the thin films can modulate one or properties of cells in the wound environment or in the immediate vicinity of the wound. The properties that are modulated, e.g., are increased or decreased, include, but are not limited to adhesion, migration, proliferation, differentiation, extracellular matrix secretion, phagocytosis, MMP activity, contraction, and combinations thereof. The thin films described herein can be covered with a secondary dressing, or bandage, if it is desired to protect the layer or to provide additional moisture absorption.

Free-standing thin films described herein can be applied to a moist wound and bandaged over, where the oxidative nature of the film is beneficial in providing reactive oxygen species (ROS), optionally providing desired oxidative potentials (peroxide concentrations) during different stages (e.g., of wound healing).

Free-standing thin films can be applied to dry skin affected by hyperkeratosis (including dermatitis, psoriasis, eczema, corns, calluses, ingrown nails, etc.) and either moistened or simply bandaged over whereby an additive, such as urea, can act as a humectant to draw moisture to the site. The thin films can also be used as a skin-contact layer to a complement a wound care product, for example a disposable bandage.

The peroxide and/or other additive can be loaded at a concentration of approximately 0.01 to 100 μg/cm2 in the hydrophilic polymer layer. The peroxide and/or other additive can be provided in hydrophilic polymer layer in an amount so that the peroxide and/or other additive is released at a rate of about 0.01 to 100 μg/cm2 per day. The peroxide and/or other additive can be provided in the hydrophilic polymer layer in an amount so that the peroxide and/or other additive is released at a rate of about 0.01 to 100 μg/cm2 per day for up to 5, 10, 20, 25, or 30 days. The peroxide and/or other additive can be provided in the second polymer layer at a concentration of approximately 0.01 μg/cm2 to 10 mg/cm2. The peroxide and/or other additive can be provided in the second polymer layer in an amount so that the peroxide and/or other additive is released at a rate of about 0.01 μg/cm2 to 10 mg/cm2 per day. The peroxide and/or other additive can be provided in the second polymer layer in an amount so that the peroxide and/or other additive is released at a rate of about 0.01 μg/cm2 to 10 mg/cm2 per day for up to 5, 10, 20, 25, or 30 days.

Various features and embodiments of the disclosure are illustrated in the following representative examples, which are intended to be illustrative, and not limiting. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1: Making a PVA Thin Film with Peroxide and Urea

A 20 wt % solution of Poval 4-98 polyvinyl alcohol is prepared by mixing 33.12 kg of dry Poval 4-98 granules, from Kuraray, into deionized water under constant agitation, in order to fully wet all granules individually. The dispersion is heated to 90° C. to fully dissolve the PVA granules. The solution is then allowed to cool to room temperature. The solution is cooled to 30° C. or less before further processing.

Carbamide peroxide, from Aldrich (p/n 289132) is then added in pellet form up to 40 wt % of the final solution. 66.24 kg of carbamide peroxide was used. The pellets are dissolved and no gas evolution due to peroxide degradation was observed in the vessel. This solution is pumped into the pan of a gravure coater (custom machine from SMBK (https://smbk.de/)) by means of a positive displacement transfer pump, and applied via direct gravure to a passing 5 mil PET substrate which has been pretreated with silicone release, commercially available from Saint-Gobain Performance Plastics as Versiv 8310, at a rate of 160 cm3/m2. The wet film is then dried in a convection drier, and wrapped under tension for storage. Sheets can then be peeled free of the carrier substrate as needed.

The reduction potential of these films over time was tested by applying the thin films to a surface containing a silver nanoparticle film, and qualitatively observing oxidation of the silver particles over the next 20-30 seconds. With storage conditions protecting the thin film from light and heat, the thin films retained their oxidative potential over 2 months.

### Example 2: Making a PVA Thin Film with Peroxide

A solution of fully hydrolyzed PVA (98% degree of hydrolysis) was made in an aqueous solution at high temperature (e.g., 70-90° C.). PVAs and conditions for making suitable solutions are described in the Clariant Mowoil Manual, found at ia903101.us.archive.org/35/items/polyvinylalcoholmanufacturemanual/Mowiol % 20manual.p df, which is incorporated by referenced in its entirety for all purposes.

The stock solution of PVA dissolved and used immediately without exceeding the following maximum concentrations:
Mowiol 3-98: 30%
Mowiol 4-98: 25%
Mowiol 6-98: 25%
Mowiol 10-98: 20%
Mowiol 20-98: 15%
Mowiol 56-98: 12%
Mowiol 28-99: 12%

PVA granules are dissolved at 70-90° C., and the solution is allowed to cool to room temperature prior to adding a peroxide source. The peroxide can be a liquid (e.g., hydrogen peroxide) or solid (peroxide-urea adduct, sodium percarbonate, calcium peroxide, magnesium peroxide). Peroxide-urea is added. The solid adduct of peroxide-urea can be dissolved in the PVA solution (e.g., 20% solution of Mowiol 4-98 PVA up to 40% of the total solution). This solution is applied to a suitable release liner (e.g., silicone treated PET) by gravure, smooth-roll, slot-die, spin-coating, draw-down bar, or other wet film coating technique, followed by forced convection drying, either with or without additional heat, to reduce the moisture content of the film from ˜80% to less than 12% (e.g., 5-8%). The PVA-peroxide-urea layer can then be packaged to prevent excessive drying or ambient light either before or after peeling it off the release liner backing.

Final dry PVA films are made having 25% hydrogen peroxide by weight, 41% urea by weight and 33% PVA by weight have been made. The final dry PVA film can have 5-25% hydrogen peroxide by weight, 8-41% urea by weight, and 33-86% PVA by weight. The final dry PVA film can have 3-25% hydrogen peroxide by weight, 5-41% urea by weight, and 33-91% PVA by weight. The peroxide can have anti-microbial effects (e.g., killing or inhibiting growth) against microorganisms, and the urea can also have antimicrobial effects. Urea can used as a humectant and/or moisturizer to promote rehydration of the skin and replenish the urea content lost from cleansing of the skin.

Final dry PVA films have retained their oxidative capacity for weeks to 2 months (i.e., the peroxide remains stable for weeks to 2 months).

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the scope of the invention(s) of the disclosure.

1. A thin film comprising: a hydrophilic polymer, a urea, a peroxide, and water.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A method comprising: making an aqueous solution of a hydrophilic polymer at a high temperature; cooling the aqueous solution of the hydrophilic polymer; adding a peroxide to the aqueous solution of the hydrophilic polymer; coating the aqueous solution of hydrophilic polymer onto a substrate having a release coated surface; drying the hydrophilic polymer on the substrate; and peeling a thin film of the hydrophilic polymer from the release coated surface of the substrate.

22. The method of claim 21, wherein the thin film is comprised of 34-99% hydrophilic polymer by weight, and 1-66% hydrogen peroxide by weight.

23. (canceled)

24. (canceled)

25. The method of claim 21, wherein the hydrophilic polymer is a GRAS hydrophilic polymer.

26. The method of claim 25, wherein the GRAS hydrophilic polymer is selected from the group consisting of: a polyvinyl alcohol, a chitosan, a starch, an alginate, a dextran, a dextrin, a chitin, a guar gum, a gum karaya, agar, a Fenugreek seed mucilage, a Soy polysaccharide, a Gellan gum, a Mango peel pectin, a Lepidium sativum mucilage, a Plantago ovata seed mucilage, an Aegle marmelos gum, a Locust bean gum, a Lepidium sativum, a Mangifera indica gum, a Hibiscus rosa-sinensis mucilage, a carrageenan, a hyaluronic acid, a carboxymethylcellulose, a carnauba wax, a carob bean gum, a carotene, a cellulose, a gelatin, a gum Arabic, a gum Ghatti, a gum gualac, a gum tragacanth, a hydroxypropylmethyl cellulose, a methylcellulose, a polyethylene glycol, and a propylene glycol.

27. The method of claim 25, wherein the GRAS hydrophilic polymer is a polyvinyl alcohol.

28. The method of claim 25, wherein the GRAS hydrophilic polymer is a carrageenan.

29. The method of claim 25, wherein the GRAS hydrophilic polymer is a hyaluronic acid.

30. The method of claim 25, wherein the GRAS hydrophilic polymer is a carboxymethylcellulose.

31. The method of claim 25, wherein the GRAS hydrophilic polymer is a cellulose.

32. The method of claim 25, wherein the GRAS hydrophilic polymer is a hydroxypropylmethyl cellulose.

33. The method of claim 25, wherein the GRAS hydrophilic polymer is a methylcellulose.

34. The method of claim 25, wherein the GRAS hydrophilic polymer is a polyethylene glycol.

35. (canceled)

36. The method claim 21, further comprising an additive.

37. The method of claim 36, wherein the additive is an antibiotic, a salicylic acid, a hydroquinone, a retinoid, a hyaluronic acid, or a vitamin C.

38. The method of claim 36, wherein the additive is an antibiotic.

39. The method of claim 38, wherein the antibiotic is an aminoglycoside, a sulfonamide, a tetracycline, a carbapenem, a cephalosporin, a 2-quinolone, a 4-quinolone, a glycopeptide, a penicillin, a rifamycin, a monobactam, an oxazolidinone antibiotic, a streptogramin, or a polypeptide antibiotic.

40. The method of claim 36, wherein the additive is a trophic factor, an extracellular matrix component, an ECM fragment, an enzyme, an enzyme inhibitor, a defensin, an antimicrobial, an antiviral, an antifungal, a buffering agent, a vitamin, an anticoagulant, a coagulation factor, an anti-inflammatory agent, a vasoconstrictor, a vasodilator, a diuretic, or an anti-cancer agent.

41. The method of claim 22, wherein the peroxide is hydrogen peroxide, a calcium peroxide, a sodium peroxide, a lithium peroxide, a barium peroxide, a magnesium peroxide, a zinc peroxide, a peroxide-urea adduct, a carbamide peroxide, or a benzoyl peroxide.

42. The method of claim 23, wherein the peroxide is a peroxide-urea adduct.
